(19) United States
(12) Patent Application Publication
(10) Pub. No.: US 2013/0210079 A1
(43) Pub. Date: Aug. 15, 2013

(54) REAGENTS AND METHODS FOR AUTOLIGATION CHAIN REACTION

(71) Applicants: Dusan Stanojevic, Arlington, MA (US); Ricardo Mancebo, Fremont, CA (US)

(72) Inventors: Dusan Stanojevic, Arlington, MA (US); Ricardo Mancebo, Fremont, CA (US)

(21) Appl. No.: 13/727,174

(22) Filed: Dec. 26, 2012

Related U.S. Application Data
(60) Provisional application No. 61/580,998, filed on Dec. 28, 2011.

(51) Int. Cl. C12P 19/34

(52) U.S. Cl. CPC C12P 19/34
USPC 435/91.2; 536/24.33

(57) ABSTRACT

The invention relates to the exponential amplification of specific target nucleic acids. The invention provides methods, reagents and kits for carrying out such exponential amplification via the autoligation chain reaction (ACR).

### RELATED APPLICATIONS

This application claims the benefit of U.S. Provisional Application No. 61/580,998, filed on Dec. 28, 2011. The entire teachings of the above application are incorporated herein by reference.

### GOVERNMENT FUNDING

This invention was made in part with government support under grant #1046508 awarded to the inventors by the National Science Foundation. The government has certain rights in the invention.

### BACKGROUND OF THE INVENTION

Amplification of nucleic acid sequences is a widespread technology that has been used for many purposes, including diagnostic and forensic testing. Currently, this is carried out using polymerase chain reaction (PCR). Unfortunately, critical barriers exist with PCR that prevent both clinical and research labs from adopting PCR-based assays into a routine setting, due to bottlenecks with sample preparation and assay development costs. Specifically, the PCR inhibitors, such as inhibitors to polymerases, found in many laboratory samples and clinical specimens cause low sensitivity and false-negative results in clinical and forensic tests that rely on PCR-based molecular techniques. Therefore, it is widely accepted that purification or pre-amplification of target DNA nucleic acids is required to remove or dilute out inhibitors prior to PCR amplification to obtain successful results. Optimization of PCR for genetic testing with different sample types can be labor intensive, requiring extensive amounts of upfront development work, which in turn can significantly increase both the overall cost of a test and the time-to-result.1-5 With the upsurge in genetic information and the resultant increase in DNA biomarkers, researchers are now seeking new technologies to rapidly and cost-effectively interrogate this new information in a routine setting. However, the critical barriers associated with PCR make this technology too cost-prohibitive and too labor-intensive to use as a testing method for price-sensitive laboratories with limited resources and large numbers of samples.

Recently, technology has been developed to detect and monitor cellular genetic mutations using RNA-templated chemistry, in which chemically modified probes fluoresce when they hybridize to their genetic target in intact bacterial and human cells.10-15 This probe-based strategy, called quenched autoligation (QUAL), utilizes two self-reacting oligonucleotide probes that provide a fluorescence signal in the presence of fully complementary nucleic acid target sequence. A first oligonucleotide having a 3′-phosphoromono-thioate nucleophilic group anneals to a template target sequence, such that the 3′-phosphoromono-thioate nucleophilic group is juxtaposed to a 5′-electrophilic dabsylated group quencher of a second annealed oligonucleotide which has a fluorescein group quenched by the dabsyl group. This tandem configuration along a DNA template catalyzes the autoligation reaction, and joins the two oligonucleotides into a single probe. Upon ligation, the dabsyl quencher is displaced, and the fluoresceinyl fluorophore becomes un-quenched, resulting in an increase in fluorescence signal.

These short QUAL probes have been used to distinguish closely related bacterial species by discriminating single nucleotide differences in 16S rRNA sequences within live cells. QUAL offers the potential to develop new bioanalytical assays in living cells, such as RNA localization, transcription, and RNA processing. However, this strategy is not compatible with in vitro applications that require the detection of small amounts of double-stranded nucleic acid sequences that are typically found in samples used for routine genetic testing of DNA biomarkers. For example, a QUAL in vitro reaction typically contains 10¹³ copies of single-stranded oligo DNA template, but a routine molecular assay can contain 10³ or fewer copies of dsDNA biomarkers—a ten billion-fold difference in copy-number detection. Detecting such a small number of molecules requires amplification of those numbers, which in turn requires thermal denaturation of the double stranded DNA molecules. Unfortunately, the autoligation chemistries used in QUAL are not thermostable enough to last more than a few minutes at the high temperatures needed to separate double-stranded DNA. Thus, these procedures are not suitable for amplifying nucleic acid sequences.

There is, therefore, a need for thermostable reagents and methods for amplifying nucleic acid sequences without enzymes or nucleosides to enable cost-effective and easier-to-use alternatives for genetic testing that can be implemented in routine settings across multiple sample types without any sample-prep development.

### SUMMARY OF THE INVENTION

The invention relates to amplification of nucleic acid sequences. More particularly, the invention relates to amplification of nucleic acid sequences without enzymes or nucleosides. The invention provides thermostable reagents and methods for amplifying nucleic acid sequences without enzymes or nucleosides. In a first aspect, the invention provides a method for exponentially amplifying a specific target nucleic acid sequence. The method according to this aspect of the invention comprises contacting the target nucleic acid sequence with a first forward primer nucleic acid, a second forward primer nucleic acid, a first reverse primer nucleic acid and a second reverse primer nucleic acid under conditions wherein the primer nucleic acids specifically anneal with the target nucleic acid sequence. One forward primer nucleic acid has a thermally stable first bond-forming reactive moiety and the other forward primer nucleic acid has a thermally stable second bond-forming reactive moiety. One reverse primer nucleic acid has a thermally stable first bond-forming reactive moiety and the other reverse primer nucleic acid has a thermally stable second bond-forming reactive moiety. The first forward primer nucleic acid and the second forward primer nucleic acid are annealed to the target nucleic acid sequence such that the reactive moiety of the first forward primer nucleic acid and the reactive moiety of the second forward primer nucleic acid are juxtaposed. The first reverse primer nucleic acid and the second reverse primer nucleic acid are annealed to the target nucleic acid sequence such that the reactive moiety of the first reverse primer nucleic acid and the reactive moiety of the second reverse primer nucleic acid are juxtaposed. The reactive moiety of the first forward primer nucleic acid forms a chemical bond with the reactive moiety of the second forward primer nucleic acid to form a first ligation product, and the reactive moiety of the first reverse primer nucleic acid forms a chemical bond with the reactive moiety of the second reverse primer nucleic acid to form a second ligation product. Thus, the first ligation product forms a duplex with the target nucleic acid sequence and the second ligation product forms a duplex with the target nucleic acid sequence. The duplexes are then thermally disrupted to form target nucleic acid sequences and the steps are repeated to exponentially amplify the target nucleic acid sequences.

In some embodiments, the thermally stable first bond-forming reactive moiety is a nucleophilic moiety and the thermally stable second bond-forming reactive moiety is an electrophilic moiety. In some embodiments, the thermally stable first bond-forming reactive moiety is an electrophilic moiety and the thermally stable second bond-forming reactive moiety is a nucleophilic moiety.

In some embodiments, one forward or reverse primer nucleic acid comprises a dye or detectable group. In some embodiments, one forward or reverse primer nucleic acid comprises a fluorescence resonance energy transfer (FRET) donor fluorophore and/or the other forward or reverse primer nucleic acid comprises a FRET acceptor fluorophore, and the ligation products are detected by FRET.

In a second aspect, the invention provides reagents for exponentially amplifying a target nucleic acid sequence. In some embodiments, a reagent according to the invention comprises a first forward primer nucleic acid having a thermally stable first bond-forming reactive moiety. In some embodiments, a reagent according to the invention comprises a second forward primer nucleic acid having a thermally stable second bond-forming reactive moiety. In some embodiments, a reagent according to the invention comprises a first reverse primer nucleic acid having a thermally stable first bond-forming reactive moiety. In some embodiments, a reagent according to the invention comprises a second reverse primer nucleic acid having a thermally stable second bond-forming reactive moiety. In such embodiments, the first bond-forming reactive moiety forms a chemical bond with the second bond-forming reactive moiety, when the first forward primer nucleic acid and the second forward primer nucleic acid are juxtaposed by annealing with a target nucleic acid and when the first reverse primer and the second reverse primer nucleic acid are juxtaposed by annealing with a target nucleic acid. In some embodiments, the first bond-forming reactive moiety is a nucleophilic moiety and the second bond-forming reactive moiety is an electrophilic moiety. In some embodiments, the first bond-forming reactive moiety is an electrophilic moiety and the second bond-forming reactive moiety is a nucleophilic moiety. In some embodiments, one forward or reverse primer nucleic acid comprises a dye or detectable group. In some embodiments, one forward or reverse primer nucleic acid comprises a FRET donor fluorophore and/or the other forward or reverse primer nucleic acid comprises a FRET acceptor fluorophore.

In a third aspect, the invention provides a kit for exponentially amplifying a target nucleic acid sequence. The kit according to this aspect of the invention comprises a first forward primer nucleic acid, a second forward primer nucleic acid, a first reverse primer nucleic acid, and a second reverse primer nucleic acid. In the kit according to this aspect of the invention, the first forward primer nucleic acid, the second forward primer nucleic acid, the first reverse primer nucleic acid, and the second reverse primer nucleic acid are as described for the second aspect according to the invention.

### BRIEF DESCRIPTION OF THE DRAWINGS

The foregoing will be apparent from the following more particular description of example embodiments of the invention, as illustrated in the accompanying drawings.

FIG. 1 illustrates the strategy and expected results from two rounds of thermocycling with Autoligation Chain Reaction (ACR), in which four double-stranded products are generated from the exponential amplification of a single target sequence.

FIG. 2 shows an example of chemistry development of bond-forming reactive moieties for ACR. A) Synthesis of primer nucleic acid nucleophiles containing a 3′-thio thymidine nucleophile monomer according to the method described in^{7,8}, with a modification to include a non-standard amino-modifier residue (2′-amino-dT) that carries a short alkyl linker with no double bonds. B) Preparation of the solid support. C) Synthesis of primer nucleic acid electrophiles containing alkyl-halide or maleimide groups incorporated at the 5′ end.

FIG. 3 shows an example spectrofluorimeter readout for primer nucleic acids labeled with FAM and Texas Red.

FIG. 4 shows unstained and stained polyacrylamide gels after an autoligation reaction with a first forward primer nucleic acid containing a first bond-forming reactive moiety and a second forward primer nucleic acid containing a second bond-forming reactive moiety in which the second forward primer is labeled with FAM.

FIG. 5 shows a stained polyacrylamide gel after an autoligation reaction with a first reverse primer nucleic acid containing a first bond-forming reactive moiety and a second reverse primer nucleic acid containing a second bond-forming reactive moiety in which the first and second reverse primers are unlabeled.

FIG. 6 shows thermal-stability of bond-forming reactive moieties on first and second forward primer nucleic acids in which the second forward primer nucleic acid contains FAM and FAM fluorescence of the autoligation reaction is detected on an unstained 20% acrylamide+urea denaturing gel using the Typhoon Trio+ imaging system.

FIG. 7 shows a comparison between thermal-stabile bond-forming reactive moieties comprised of a thio thymidine nucleophile and a bromoacetyl electrophile versus thermal-unstable bond-forming reactive moieties comprised of a phosphoromono-thioate ester nucleophile and a dabsylate electrophile on an unstained 20% acrylamide+urea denaturing gel.

FIG. 8 shows FAM/Texas Red FRET fluorescence of ACR reactions on an unstained 20% acrylamide+urea denaturing gel using the Typhoon Trio+ imaging system.

FIG. 9A shows real-time amplification plots on the LightCycler® 480 II using FAM/Texas Red FRET fluorescence in ACR reactions to demonstrate exponential amplification. FIG. 9B shows the same reaction products run on a SYBR Green I stained 20% acrylamide+urea denaturing gel using the Typhoon Trio+ imaging system.

FIG. 10 shows FAM/Texas Red FRET fluorescence in ACR reactions with decreasing amounts of template on an unstained 20% acrylamide+urea denaturing gel using the Typhoon Trio+ imaging system.

### DETAILED DESCRIPTION OF THE INVENTION

A description of example embodiments of the invention follows.

The invention relates to amplification of nucleic acid sequences. More particularly, the invention relates to amplification of nucleic acid sequences without enzymes or nucleosides. The invention provides thermostable reagents and methods for amplifying nucleic acid sequences without enzymes or nucleosides.

In a first aspect, the invention provides a method for exponentially amplifying a specific target nucleic acid sequence. The method according to this aspect of the invention comprises contacting the target nucleic acid sequence with a first forward primer nucleic acid, a second forward primer nucleic acid, a first reverse primer nucleic acid and a second reverse primer nucleic acid under conditions wherein the primer nucleic acids specifically anneal with the target nucleic acid sequence. One forward primer nucleic acid has a thermally stable first bond-forming reactive moiety and the other forward primer nucleic acid has a thermally stable second bond-forming reactive moiety. One reverse primer nucleic acid has a thermally stable first bond-forming reactive moiety and the other reverse primer nucleic acid has a thermally stable second bond-forming reactive moiety. The first forward primer nucleic acid and the second forward primer nucleic acid are annealed to the target nucleic acid sequence such that the reactive moiety of the first forward primer nucleic acid and the reactive moiety of the second forward primer nucleic acid are juxtaposed. The first reverse primer nucleic acid and the second reverse primer nucleic acid are annealed to the target nucleic acid sequence such that the reactive moiety of the first reverse primer nucleic acid and the reactive moiety of the second reverse primer nucleic acid are juxtaposed. The reactive moiety of the first forward primer nucleic acid forms a chemical bond with the reactive moiety of the second forward primer nucleic acid to form a first ligation product, and the reactive moiety of the first reverse primer nucleic acid forms a chemical bond with the reactive moiety of the second reverse primer nucleic acid to form a second ligation product. Thus, the first ligation product forms a duplex with the target nucleic acid sequence and the second ligation product forms a duplex with the target nucleic acid sequence. The duplexes are then thermally disrupted to form target nucleic acid sequences and the steps are repeated to exponentially amplify the target nucleic acid sequences.

In some embodiments, the thermally stable first bond-forming reactive moiety is a nucleophilic moiety and the thermally stable second bond-forming reactive moiety is an electrophilic moiety. In some embodiments, the thermally stable first bond-forming reactive moiety is an electrophilic moiety and the thermally stable second bond-forming reactive moiety is a nucleophilic moiety.

In some embodiments, one forward or reverse primer nucleic acid comprises a dye or detectable group. In some embodiments, one forward or reverse primer nucleic acid comprises a FRET donor fluorophore and/or the other forward or reverse primer nucleic acid comprises a FRET acceptor fluorophore, and the ligation products are detected by FRET.

In a second aspect, the invention provides reagents for exponentially amplifying a target nucleic acid sequence. In some embodiments, a reagent according to the invention comprises a first forward primer nucleic acid having a thermally stable first bond-forming reactive moiety. In some embodiments, a reagent according to the invention comprises a second forward primer nucleic acid having a thermally stable second bond-forming reactive moiety. In some embodiments, a reagent according to the invention comprises a first reverse primer nucleic acid having a thermally stable first bond-forming reactive moiety. In some embodiments, a reagent according to the invention comprises a second reverse primer nucleic acid having a thermally stable second bond-forming reactive moiety. In such embodiments, the first bond-forming reactive moiety forms a chemical bond with the second bond-forming reactive moiety, when the first forward primer nucleic acid and the second forward primer nucleic acid are juxtaposed by annealing with a target nucleic acid and when the first reverse primer and the second reverse primer nucleic acid are juxtaposed by annealing with a target nucleic acid. In some embodiments, the first bond-forming reactive moiety is a nucleophilic moiety and the second bond-forming reactive moiety is an electrophilic moiety. In some embodiments, the first bond-forming reactive moiety is an electrophilic moiety and the second bond-forming reactive moiety is a nucleophilic moiety. In some embodiments, one forward or reverse primer nucleic acid comprises a dye or detectable group. In some embodiments, one forward or reverse primer nucleic acid comprises a FRET donor fluorophore and/or the other forward or reverse primer nucleic acid comprises a FRET acceptor fluorophore. In a third aspect, the invention provides a kit for exponentially amplifying a target nucleic acid sequence. The kit according to this aspect of the invention comprises a first forward primer nucleic acid, a second forward primer nucleic acid, a first reverse primer nucleic acid, and a second reverse primer nucleic acid. In the kit according to this aspect of the invention, the first forward primer nucleic acid, the second forward primer nucleic acid, the first reverse primer nucleic acid, and the second reverse primer nucleic acid are as described for the second aspect according to the invention.

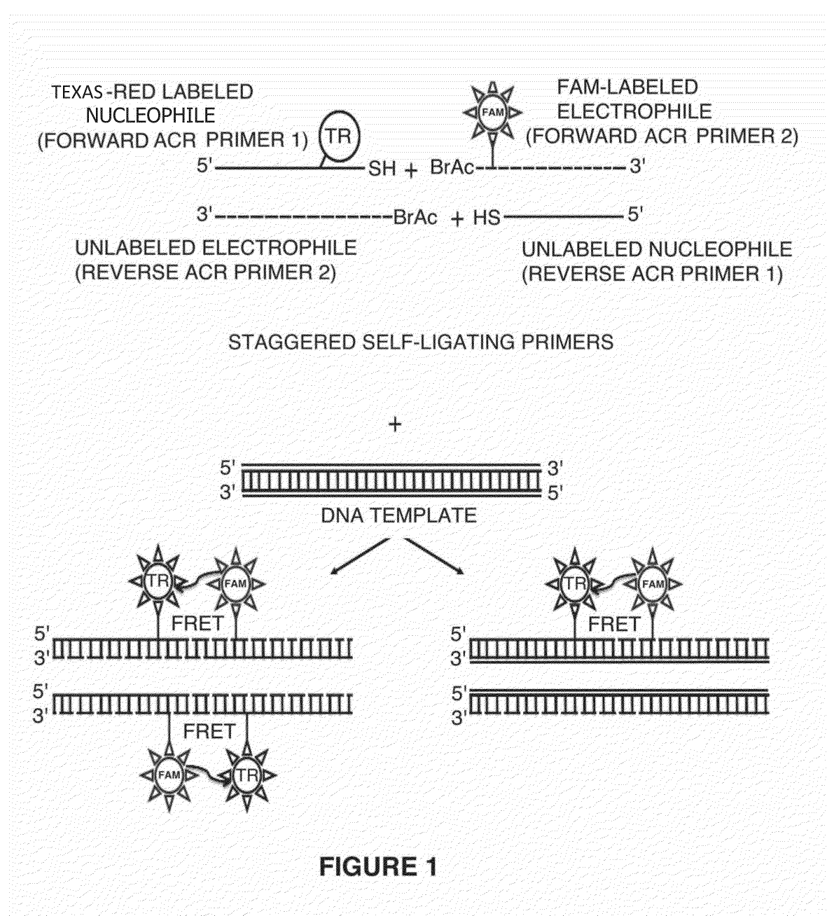

Non-limiting examples of reagents and methods according to the invention are shown in FIG. 1, which illustrates the strategy and expected results from two rounds of thermocycling with ACR, in which four double-stranded products are generated from the exponential amplification of a single target sequence. Forward ACR Primer 1 and Reverse ACR Primer 1 both contain a nucleophilic thiol moiety (SH) at the 3′ end. Forward ACR Primer 2 and Reverse ACR Primer 2 both contain an electrophilic bromoacetate moiety (BrAc) at the 5′ end. When forward and reverse primers are annealed in tandem to template, the juxtaposition of the SH and BrAc groups results in a DNA-templated autoligation reaction without any enzymes or nucleotides. Primers annealed in tandem have higher melting temperature due to stabilizing base-pair stacking interactions between the tandemly-aligned oligos.⁶ ACR is performed at annealing temperatures that favor the formation of primer/template heteroduplexes over primer dimers in homoduplexes. The resulting autoligation products are used as templates in subsequent rounds of exponential amplification.

Forward ACR Primer 1 and Reverse ACR Primer 2 and Forward ACR Primer 2 and Reverse ACR Primer 1 are complementary pairs, which increase the specificity of the reaction by sequestering the primers in duplexes until dsDNA templates outcompete the formation of oligo homoduplexes by annealing to the oligos. Because tandemly-annealed oligos on a template have significantly higher melting temperatures than individual oligos annealed to the same template, due to stabilizing base-pair stacking interactions between the tandemly-aligned oligos, ACR is performed at annealing temperatures that favor the formation of oligo/template heteroduplexes over homoduplexed oligo sets. For purposes of the invention, a “primer nucleic acid” is an oligonucleotide used in the method according to the invention to form a longer oligonucleotide via autoligation to another primer nucleic acid. Primer nucleic acids may be from about 5 to about 35 nucleotides in length. The autoligation reaction occurs when the primer nucleic acids are annealed to a target nucleic acid sequence such that a first bond-forming reactive moiety of one primer nucleic acid is juxtaposed with a second bond-forming reactive moiety of another primer nucleic acid. In some embodiments the first bond-forming reactive moiety is at a terminus (5′ or 3′) of one primer nucleic acid and the second bond-forming reactive moiety is at an opposite terminus of the other primer nucleic acid. The terms “first bond-forming reactive moiety” and “second bond-forming reactive moiety” refer to chemical functional groups that are capable of reacting with each other to form a covalent bond.

Non-limiting examples of first bond-forming reactive moieties include phosphorodithioate, phosphorotrithioate, 2′,3′-cyclic phosphate, amino-deoxyribonucleosides, thiol, amino, hydrazine and hydrazide. In certain embodiments, the first bond-forming reactive moiety is a nucleophile. A 3′-thionucleoside is a particularly preferred 3′ terminal nucleophile.

Non-limiting examples of second bond-forming reactive moieties include bromide, iodide, chloride, maleimide, dabsylate, pyridyldisulfide, tosylate, alkyne, isothiocyanate, cyclooctyne, NHS ester, imidoester, PFP ester, alkyl azide, aryl azide, isocyanate, nitrophenyl mono- or di-ester and epoxy. In certain embodiments, the second bond-forming reactive moiety is an electrophile. A 5′-bromoacetylnucleoside is a particularly preferred 5′-terminal electrophile.

Amplification of a double-stranded target nucleic acid sequence requires thermal denaturation of the target sequence. Thus, the first and second bond-forming reactive moieties must be thermally stable. “Thermally stable” means that the moiety reactivity must not be destroyed or functionally compromised at temperatures required to denature the target sequence.

In some embodiments, a dye or detectable group is used to detect the ligated products formed by annealing and autoligation. Non-limiting dyes and detectable groups include, without limitation, the groups shown in Table I below.

TABLE I
Detection Dyes and Groups

(E)-Stilbene
(Z)-Stilbene
1-Chloro-9,10-bis(phenylethynyl)anthracene
2-Chloro-9,10-bis(phenylethynyl)anthracene
2-Chloro-9,10-diphenylanthracene
5,12-Bis(phenylethynyl)naphthacene
7-Aminoactinomycin D
7-Aminoactinomycin D (7-AAD)
7-Hydroxy-4-methylcoumarin
8-Anilinonaphthalene-1-sulfonate
9,10-Bis(phenylethynyl)anthracene
Acridine orange
Acridine yellow
Alexa Fluor
Alexa Fluor 350 dye, 7-amino-4-methylcoumarin (AMC)
Alexa Fluor 405 dye
Alexa Fluor 430 dye
Alexa Fluor 488 dye
Alexa Fluor 514 dye
Alexa Fluor 532 dye
Alexa Fluor 546 dye
Alexa Fluor 555 dye
Alexa Fluor 568 dye
Alexa Fluor 594 dye
Alexa Fluor 610 dye
Alexa Fluor 633 dye
Alexa Fluor 635 dye
Alexa Fluor 647 dye
Alexa Fluor 660 dye
Alexa Fluor 680 dye
Alexa Fluor 700 dye
Alexa Fluor 750 dye
Alexa Fluor 790 dye
Allophycocyanin
ATTO dyes
Auramine-rhodamine stain
BCECF indicator
Benzanthrone
BHQ-1
BHQ-2
BHQ-3
Bimane
Blacklight paint
blue fluorescent proteins
BOBO-1, BO-PRO-1
BODIPY 630/650 dye
BODIPY 650/665 dye
BODIPY dye
BODIPY FL dye
BODIPY TMR-X dye
BODIPY TR-X dye
Brainbow
Calcein
Calcium Crimson indicator
Calcium Green indicators
Calcium Orange indicator
Carboxy SNARF indicators
Carboxyfluorescein
Carboxyfluorescein diacetate succinimidyl ester
Carboxyfluorescein succinimidyl ester
Cascade Blue dye
Cascade Yellow dye
Chemiluminescent
Colorimetric
Coumarin
Cy-3
Cy-5
Dabcyl
DAPI
Dark quencher
DDQ-I
DDQ-II
Di-8-ANEPPS, Di-4-ANEPPS
DiA
DiD (DiIC18(5))
DiI (DiIC18(3))
DiO (DiOC18(3))
DiOC6
DiR (DiIC18(7))
DyLight Fluor
Eclipse
ELF 97 alcohol
Eosin
ER Tracker Blue-White DPX
EthD-1
Ethidium bromide
excimer/exciplex partner
exciplex dyes
FAM
Fluo-3 indicator
Fluo-4
Fluo-4 indicator
FluoProbes
Fluorescein
Fluorescein isothiocyanate
Fluorescein, FITC
Fluoro-Jade stain
fluorophore-quencher couples,
FM 1-43, FM 1-43FX
FM 4-64, FM 4-64FX
Fura Red indicator
Fura-2 indicator
Fura-2-acetoxymethyl ester
gold nano particles
Green fluorescent protein
HEX
Hoechst 33258, Hoechst 33342
Indian yellow
Indo-1
inorganic quantum dots
Iowa Black FQ
Iowa Black RQ
JC-1
JC-9
JOE
LC red 640
LC red 705
Lissamine rhodamine B
Lucifer yellow
Lucifer yellow CH
Luciferin
LysoSensor Blue DND-167
LysoSensor Green DND-153, DND-189
LysoSensor Yellow/Blue DND-160 (PDMPO)
LysoTracker Green
LysoTracker Red
Magnesium Green indicator
Marina Blue dye
Merocyanine
MGB groups
MitoTracker Green FM
MitoTracker Orange CMTMRos
MitoTracker Red CMXRos
Monobromobimane
NBD amines
NED
NeuroTrace 500/525 green-fluorescent Nissl stain
Nile blue
Nile red
Optical brightener
Oregon Green 488 dye and Oregon Green 488 BAPTA indicators
Oregon Green 514 dye
Pacific Blue dye
Pacific Orange dye
Perylene
Phloxine
Phycobilin
Phycoerythrin
Phycoerythrobilin
POPO-1, PO-PRO-1
Propidium iodide
Pyranine
QSY-21
QSY-7
R-phycoerythrin
red fluorescent proteins
Resorufin
RH 414
Rhod-2 indicator
Rhodamine
Rhodamine 110
Rhodamine 123
Rhodamine 123
Rhodamine 6G
Rhodamine Green dye
Rhodamine Red dye
RiboGreen
RoGFP
ROX
Rubrene
SERRS-active fluorescence dyes
Sodium Green indicator
Sulforhodamine 101
Sulforhodamine B
SYBR Green
Synapto-pHluorin
SYTO blue-fluorescent nucleic acid stains 40, 41, 42, 43
SYTO blue-fluorescent nucleic acid stains 44, 45
SYTO green-fluorescent nucleic acid stains 11, 14, 15, 20, 22, 25
SYTO green-fluorescent nucleic acid stains 12, 13, 16, 21, 23, 24
SYTO orange-fluorescent nucleic acid stains 80, 81, 82, 83
SYTO orange-fluorescent nucleic acid stains 84, 85
SYTO red-fluorescent nucleic acid stains 17, 59, 61, 64
SYTO red-fluorescent nucleic acid stains 60, 62, 63
SYTOX Blue nucleic acid stain
SYTOX Green nucleic acid stain
SYTOX Orange nucleic acid stain
TAMRA
TET
Tetramethylrhodamine, Rhodamine B
Tetraphenyl butadiene
Tetrasodium tris(bathophenanthroline disulfonate)ruthenium(II)
Texas Red
Texas Red-X dye
Titan yellow
TMR
TOTO-1, TO-PRO-1
TOTO-3, TO-PRO-3
TSQ
Umbelliferone
X-rhod-1 indicator
Yellow fluorescent protein
YOYO-1, YO-PRO-1
YOYO-3, YO-PRO-3

In some embodiments, the first forward primer and second forward primer or the first reverse primer are conjugated to dyes that are, respectively, a donor dye and an acceptor dye for fluorescence resonance energy transfer (FRET). Alternatively, the first forward primer and second forward primer or the first reverse primer are conjugated to dyes that are, respectively, an acceptor dye and a donor dye for FRET. Alternatively, the donor and acceptor dyes for FRET may be, respectively, on the second reverse primer and the first reverse primer or the second forward primer. Alternatively, the second reverse primer and the first reverse primer or the second forward primer are conjugated to dyes that are, respectively, an acceptor dye and a donor dye for FRET. Alternatively, the first forward primer and second forward primer are conjugated to dyes that are, respectively, an acceptor dye and a donor dye, and the second reverse primer and the first reverse primer are conjugated to dyes that are, respectively, an acceptor dye and a donor dye for FRET. Alternatively, the first forward primer and second forward primer are conjugated to dyes that are, respectively, a donor dye and an acceptor dye, and the second reverse primer and the first reverse primer are conjugated to dyes that are, respectively, a donor dye and an acceptor dye for FRET. In some embodiments, the donor and acceptor dyes are spaced from about 5 to about 10 nucleotides apart within the autoligation product. In a particularly preferred embodiment, the donor dye is FAM and the acceptor dye is Texas Red.

In some embodiments, the dye or detectable group is quenched by a quenching moiety in which annealing and autoligation separates the quenching moiety from the dye or detectable group before the ligated product is detected.

The following examples are intended to further illustrate certain embodiments of the invention and are not to be construed to limit the scope of the invention.

### Example 1

### Development of the ACR Chemistry

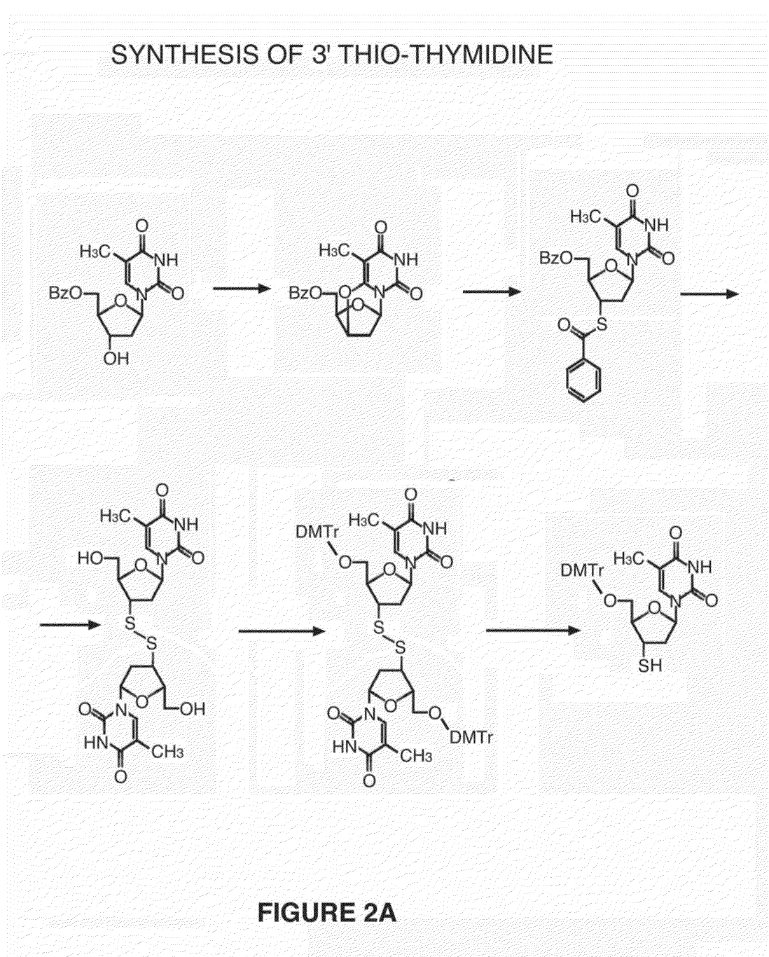

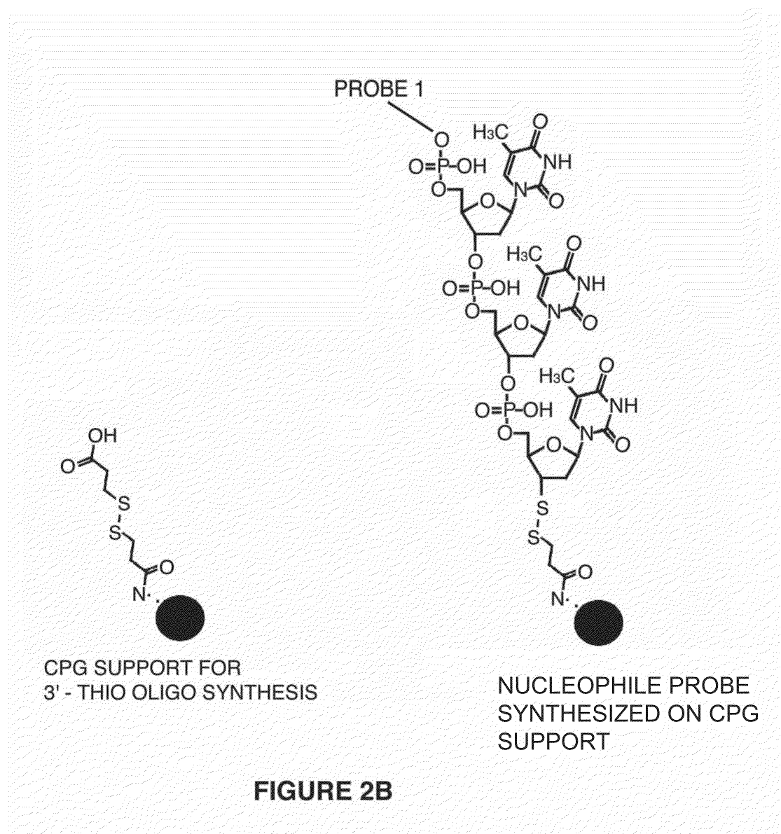

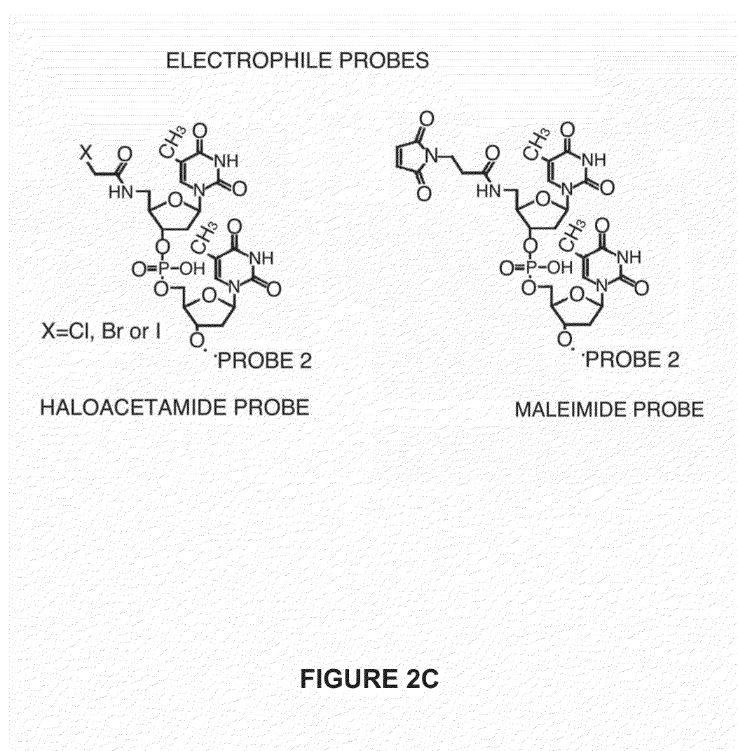

For the initial ACR chemistry we chose a system based on oligonucleotides modified with two reactive chemical groups, a thiol group incorporated at the 3′ end (i.e. nucleophile) and alkyl-halide or maleimide groups incorporated at the 5′ end (i.e. electrophile). We reasoned that this type of chemistry would allow for the nucleophile and electrophile oligos to react with each other only when juxtaposed in close vicinity by hybridizing to the complementary template.⁹ The first step in the nucleophile primer synthesis involved the preparation of a 3′-thio-2′,3′-dideoxynucleoside building block in the protected form, attached to the custom solid support via disulfide bond with a modification to include a non-standard amino-modifier residue (2′-amino-dT) that carries a short alkyl linker with no double bonds. (FIGS. 2A and 2B). This building block was incorporated at the 3′ end of nucleophilic oligonucleotide primer (FIG. 1, FIG. 2B). After deprotection, the oligonucleotide primer was released from the solid support by cleaving the disulfide bond with DTT, followed by purification. The free primer was purified by the reverse phase HPLC in pH8 tri-ethylammonium bicarbonate (buffer A) and acetonitrile (buffer B). The collected HPLC fractions were dried in lyophilizer and stored in the freezer at −20 deg. C. before use. C) The structure of 5′ electrophile oligo primers. A series of modified oligonucleotides were made by incorporating 5′-amino-dT (Glen Research) at the 5′ end via automated DNA synthesis (ChemGenes Corp), and a new custom 5′-amino-dA monomer phosphoramidite at the 5′ end via specialty DNA synthesis (ChemGenes Corp). The terminal amine group was subsequently reacted with N-hydroxysuccinimide-activated haloacetates or maleimide-group to yield three different haloacetate electrophile primers (2C, left) and a maleimide electrophile primer (2C, right). All esterification reactions were done in bicarbonate buffer pH8, at room temperature for 2 hours. The finished electrophile primers were purified and lyophilized as described above.

### Example 2

### FRET-Based Signal Detection

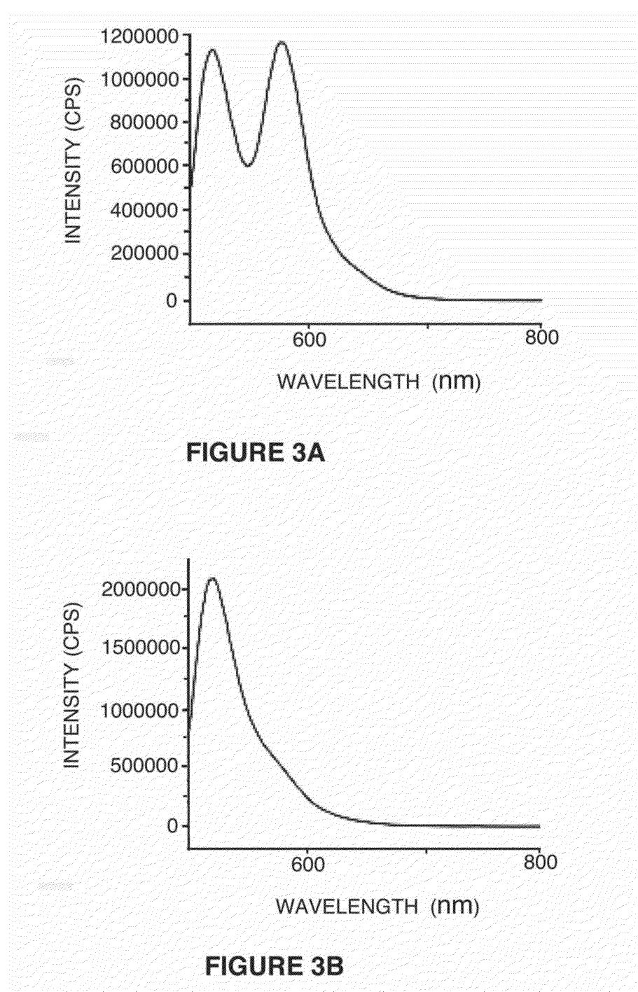

A Tx-Red labeled oligonucleotide (analogous to forward primer 1, shown in FIG. 1) and FAM-labeled oligonucleotide (analogous to forward primer 2, FIG. 1) were mixed together with unlabeled complementary single stranded oligonucleotide (analogous to the bottom strand of the DNA template shown in FIG. 1). The resulting FRET signal was measured by applying the FAM excitation wavelength of 492 nm, and observing the resulting emission spectrum between 500-800 nm. The spectrum on the left reveals the presence of the strong secondary peak at about 580 nm (FIG. 3A). FIG. 3B shows the same experiment performed in the absence of the single-stranded template (only the FAM emission peak is present at ˜520 nm). It is evident that the 580 nm peak appears only when the two labeled primers are juxtaposed in close vicinity by hybridization to the complementary single stranded template. All primers and the template were mixed in equimolar ratios (50 nM each), in TRIS pH8 buffer+200 mM potassium chloride, at room temperature. The spectra were taken in the FluoroMax 3 spectrofluorimete at room temperature. These assays have revealed that the highest FRET signal was achieved with FAM-labeled electrophile primer and Texas-Red labeled nucleophile primer, with chromophores being spaced between 5-10 bases apart within the ligation product (FIG. 3).

### Example 3

### Chemical Autoligation Reaction: Labeled Forward Primer

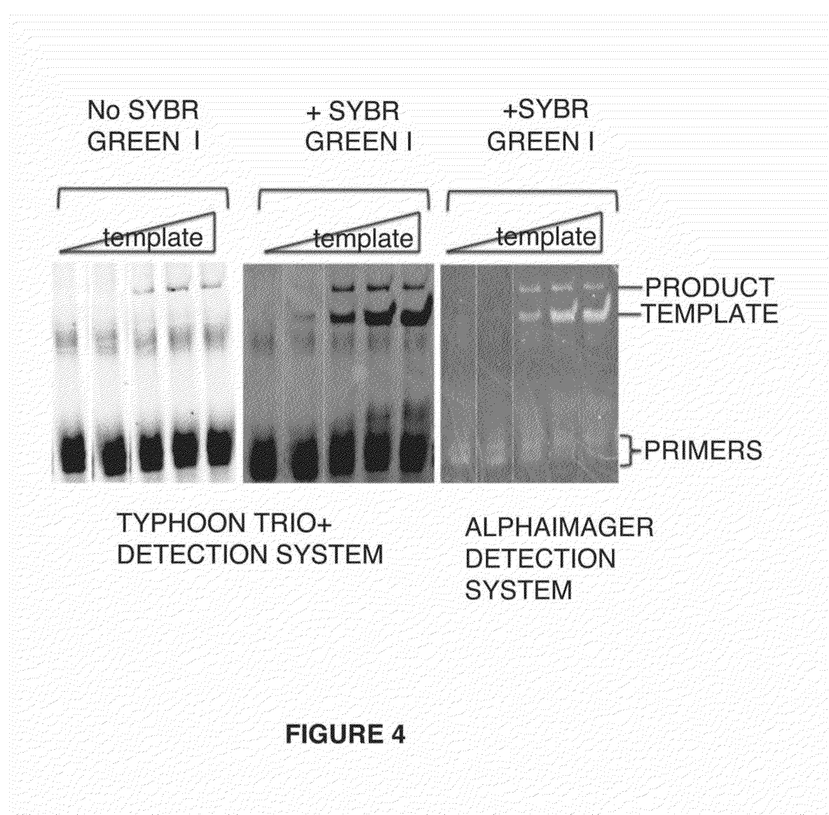

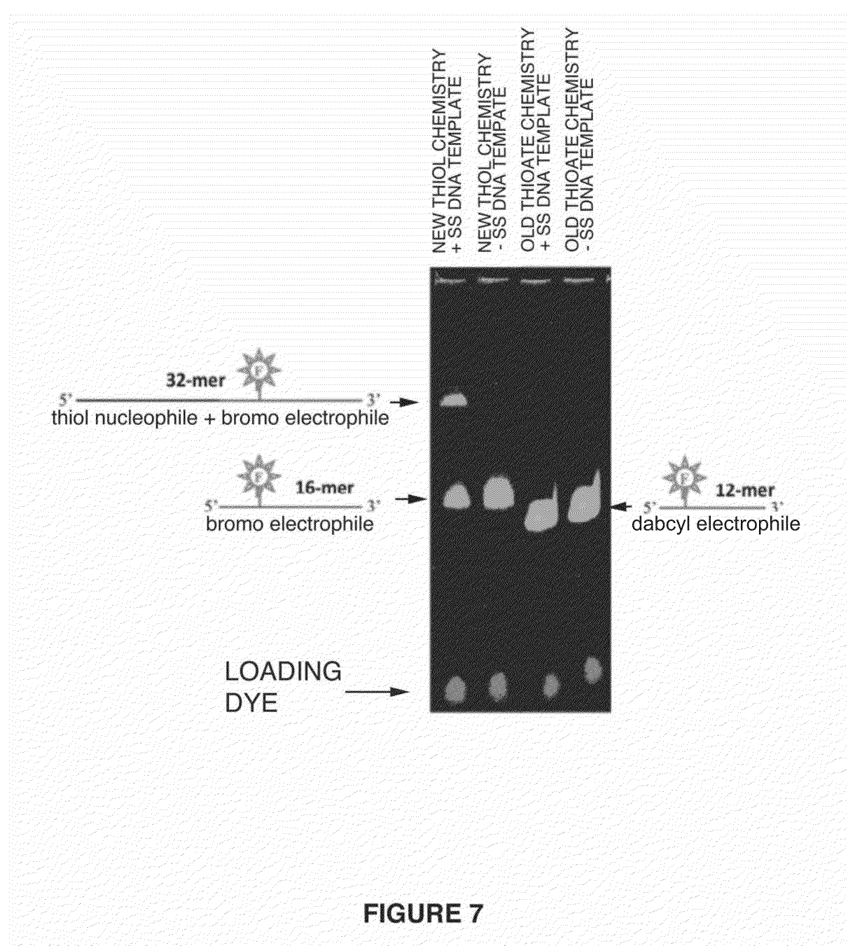

Reactions were performed using unlabeled Forward ACR Primer 1 nucleophile (GCAACGACCGTTCCGT-SH) and labeled Forward ACR Primer 2 electrophile (BrAc-TCAAT(FAM)ACTGCGCAGCC). Increasing ssDNA oligo template was added to reactions in a molar excess. Reactions were set up at room temperature and incubated at 35° C. for 20 min. Reactions were stopped with equal volumes of formamide+dye, heat denatured, cooled on ice, and load directly onto a 20% acrylamide+urea denaturing gel. The reactions worked the best at pH 7 in the presence of 20 mM DTT, at temperatures between 20-40 degrees. The most efficient autoligation was observed with Br-acetate-based electrophiles. FIG. 4 shows the efficiency of the forward ACR primers for autoligation by titrating in increasing amounts of single-stranded complementary oligo template. Lane 1 of each panel is the no-template control. The 3 panels show the same gel using different detection systems. The left panel shows FAM fluorescence using the Typhoon Trio+ imaging system. The middle panel shows SYBR fluorescence using the Typhoon Trio+ imaging system after staining the gel with SYBR Green I. The right panel shows SYBR fluorescence using the AlphaImager imaging system after staining the gel with SYBR Green I. Based on the conversion of ACR primers to ligated product observed below and in other experiments (FIG. 7 and data not shown), approximately 10%-50% of the forward primers are converted into the autoligation product.

### Example 4

### Chemical Autoligation Reaction: Unlabeled Reverse Primers

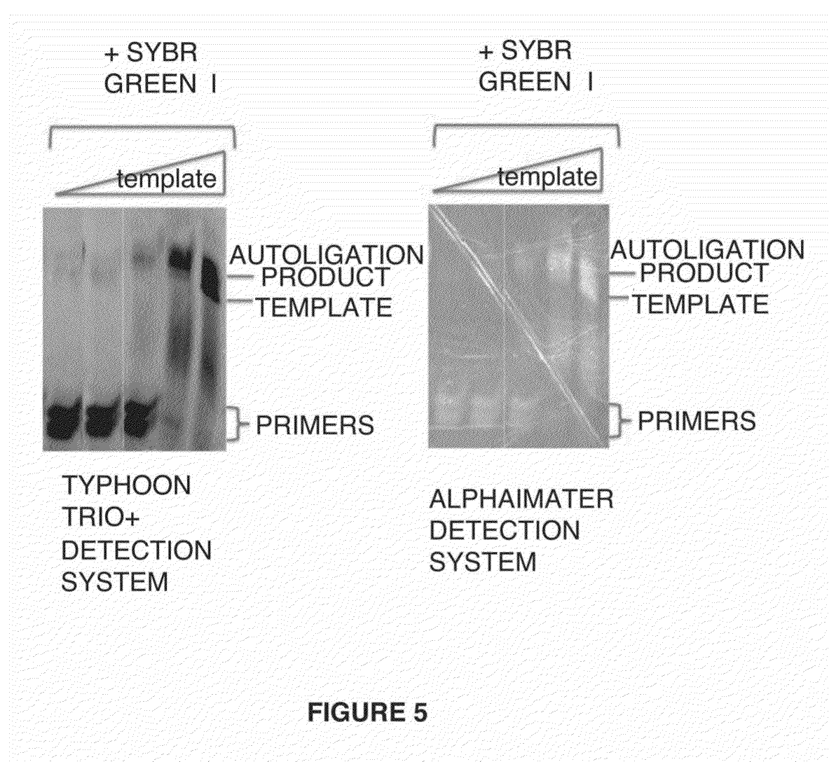

Reactions were performed using unlabeled Reverse ACR Primer 1 nucleophile (GGCTGCGCAGTAT-SH) and unlabeled Reverse ACR Primer 2 electrophile (BrAc-TGAACGGAACGGTCGTTGC). Increasing ssDNA oligo template was added to reactions in a molar excess (lanes 2-5). Lane 1 of each panel is the no-template control. Reactions were set up at room temperature and incubated at 35° C. for 20 min. Reactions were stopped with equal volumes of formamide+dye, heat denatured, cooled on ice, and load directly onto a 20% acrylamide+urea denaturing gel. FIG. 5 shows 2 panels of the same gel using different detection systems. The left panel shows SYBR fluorescence using the Typhoon Trio+ imaging system after staining the gel with SYBR Green I. The right panel shows SYBR fluorescence using the AlphaImager imaging system after staining the gel with SYBR Green I. At 17-fold molar excess of single-stranded complementary template, the autoligation reaction goes to completion and the reverse primers are converted into autoligation products.

### Example 5

### Thermostability of ACR Primers

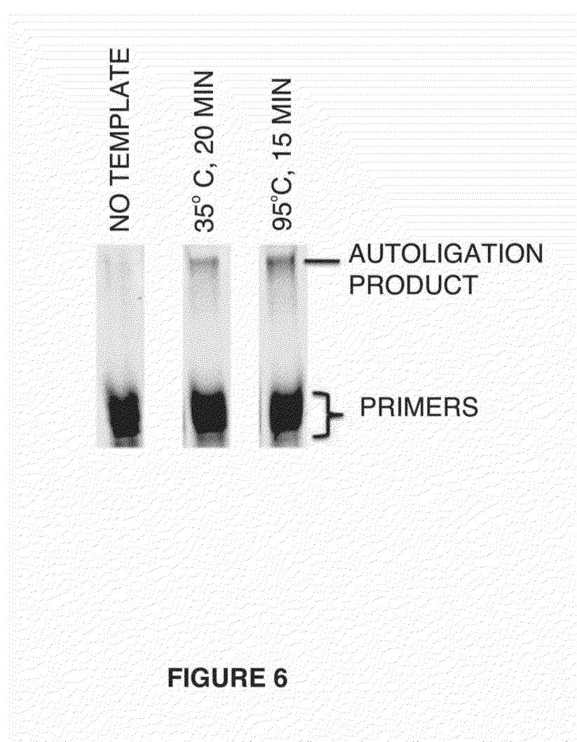

Reactions were performed using unlabeled Forward ACR Primer 1 and FAM-labeled Forward ACR Primer 2. ssDNA oligo template was added to the reactions at a 33-fold molar excess. Reactions were set up at room temperature and incubated at 35° C. for 20 min., and then thermocycled in a MultiGene Labnet thermocycler. The thermocycling protocol was 95° C. for 5 min., then 40 cycles of 95° C., 30 sec. and 20° C., 1 min. The reactions were stopped with equal volumes of formamide containing dye, heat denatured, cooled on ice, and load directly onto the denaturing gel. FIG. 6 shows FAM fluorescence of reactions on an unstained 20% acrylamide temperature (data not shown).

### Example 6

### Prior Art Comparison

Reactions were performed using ACR primers with optimized nucleophilic and electrophilic moieties using new thiol/bromoacetate chemistry according to the invention (Lanes 1 and 2), and oligo pairs previously tested with phosphoromono-thioate ester nucleophilic and dabsylate electrophilic chemistries (Lanes 3 and 4). Reactions were thermo-cycled without any enzyme or nucleotides, either in the presence (Lanes 1 and 3) or absence (Lanes 2 and 4) of complementary ssDNA oligonucleotide template. FIG. 7 shows FAM/Fluorescein fluorescence of reactions on an unstained 20% acrylamide+urea denaturing gel. The results show template-mediated thermostable autoligation with the thiol/bromoacetate chemistry but not with the phosphothioate/dabcyl chemistry. The results support the original conclusion that the phosphoromono-thioate nucleophilic moiety is not compatible with ACR due to the insufficient thermal stability.

### Example 7

### FRET Detection after Thermocycling

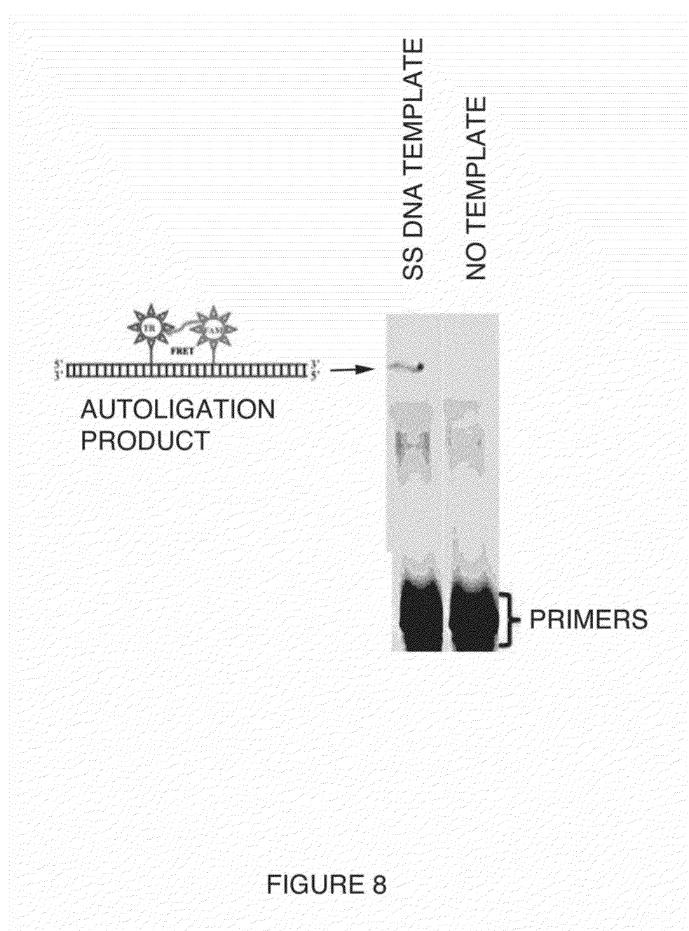

Reactions were performed using Texas Red-labeled Forward ACR Primer 1, FAM-labeled Forward ACR Primer 2, and unlabeled reverse primers, with ssDNA oligo as template. Reactions were set up on ice and thermocycled for 40 cycles. The reactions were stopped with equal volumes of formamide containing dye, heat denatured, cooled on ice, and loaded directly onto a denaturing gel. FIG. 8. The panel shows FAM/Texas Red FRET fluorescence of reactions on an unstained 20% acrylamide+urea denaturing gel using the Typhoon Trio+ imaging system. Lane 1 contains ssDNA template, and Lane 2 is the no-template control. The autoligation product was excited at 488 nM and the fluorescence emission was detected at both 520 nM (FAM channel) and 610 nM (Texas Red FRET channel) on the Typhoon Trio+ imaging system, successfully demonstrating FRET detection after thermocycling.

### Example 8

### ACR Amplification with Dual-Labeled Duplexed Tandem Primer

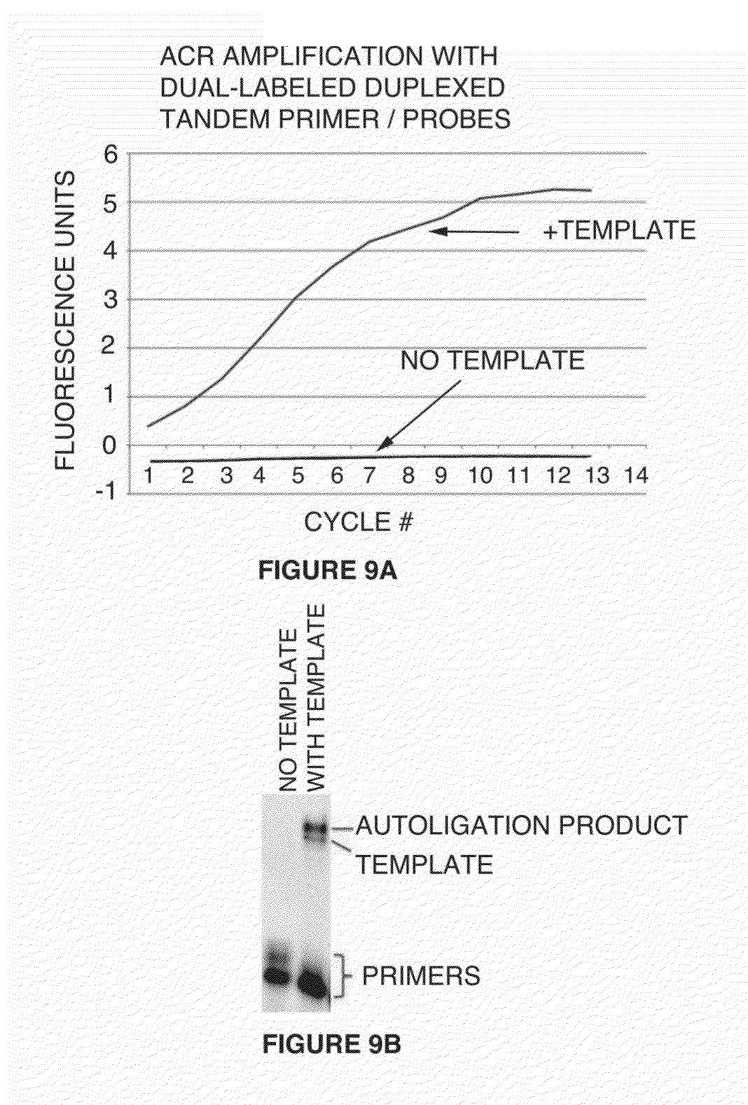

Reactions were performed using Texas Red-labeled Forward ACR Primer 1, FAM-labeled Forward ACR Primer 2, and unlabeled reverse primers, with ssDNA oligo as template. Template was added in 4-fold molar excess over the ACR primers in the reaction. Reactions were set up on ice and thermocycled for 40 cycles. The normalized baselined data was exported into Excel, and the plots were smoothed by a 4-point rolling average of the data. FIG. 9A shows real-time amplification plots on the LightCycler® 480 II using FAM/Texas Red FRET fluorescence. The trace plot in green shows exponential amplification of a reaction with template DNA, and the red plot shows a negative no-template control. Amplification was detected by cycle 2. The real-time trace shows an increase of 5 fluorescence units after baseline subtraction, with the fluorescence doubling between cycles 1 and 2, cycles 2 and 4, and cycles 3 and 5, before plateauing by cycle 10. The control reaction without template remains below the baseline. The same reactions were run on a SYBR Green I stained 20% acrylamide+urea denaturing gel using the Typhoon Trio+ imaging system (FIG. 9b). Lane 1 contains the no-template control, while Lane 2 shows the amplification product in the presence of template.

### Example 9

### Determination of Limit of Detection

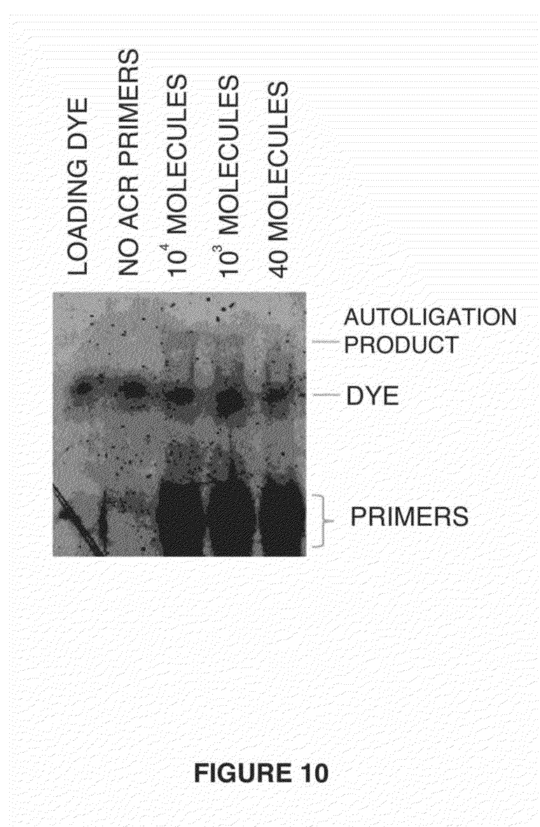

Reactions were performed using Texas Red-labeled Forward ACR Primer 1, FAM-labeled Forward ACR Primer 2, and unlabeled reverse primers, with a titration of dsDNA oligo template. The reactions were stopped with equal volumes of formamide containing dye, heat denatured, cooled on ice, and load directly onto the denaturing gel. FIG. 10 shows FAM/Texas Red FRET fluorescence of reactions on an unstained 20% acrylamide+urea denaturing gel using the Typhoon Trio+ imaging system with excitation channel 488 nm and emission channel 610 nm. Lane 3 is from 10,000 molecules, Lane 4 is from 1,000 molecules, and lane 5 is from 40 molecules of template. The band in the middle of the gel is observed in both the loading dye lane (Lane 1) and the lane with only template (Lane 2). Autoligation products are visible from reactions containing 10,000 and 1,000 molecules, but not from the reaction containing 40 molecules. The autoligation product is also not observed without template (data not shown). These results demonstrate the feasibility of exponential amplification using ACR primers.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### REFERENCES

1. Al-Soud, W. A. & Rådström, P. (2001). Purification and Characterization of PCR-Inhibitory Components in Blood Cells. Journal of Clinical Microbiology, 39(2), 485-493.
2. Huggett, J. F., Novak, T., Garson, J. A., Green, C., Morris-Jones, S. D., Miller, R. F. & Zumla, A. (2008). Differential susceptibility of PCR reactions to inhibitors: an important and unrecognised phenomenon. BMC Research Notes, 1(70), 1-9.
3. Ochert, A. S., Boulter, A. W., Birnbaum, W., Johnson, N. W. & Teo, C. G. (1994) Inhibitory effect of salivary fluids on PCR: potency and removal. Genome Res., 3, 365-368.
4. Ratnamohana, V. M., Cunningham, A. L., & Rawlinson, W. D. (1998). Removal of inhibitors of CSF-PCR to improve diagnosis of herpesviral encephalitis. Journal of Virological Methods, 72(1), 59-65.
5. Honoré-Bouakline, S., Vincensini, J. P., Giacuzzo, V., Lagrange, P. H. & Herrmann, J. L. (2003). Rapid Diagnosis of Extrapulmonary Tuberculosis by PCR: Impact of Sample Preparation and DNA Extraction. Journal of Clinical Microbiology, 41(6), 2323-2329.
6. Lane, M. J., Paner, T., Kashin, I., Faldasz, B. D., Li, B., Gallo, F. J. & Benight, A. S. (1997). The thermodynamic advantage of DNA oligonucleotide ‘stacking hybridization’ reactions: energetics of a DNA nick. Nucleic Acids Research, 25(3), 611-617.
7. Ghalia Sabbagh, Kevin J. Fettes, Rajendra Gosain, Ian A. O'Neil and Richard Cosstick (2004). Synthesis of phosphorothioamidites derived from 3′-thio-3′-deoxythymidine and 3′-thio-2′,3′-dideoxycytidine and the automated synthesis ofoligodeoxynucleotides containing a 3′-S-phosphorothiolate linkage. Nucleic Acids Research, 32(2) 495-501.
8. Meena, Mui Sam, Kathryn Pierce, Jack W. Szostak, and Larry W. McLaughlin. (2′,3′-Dideoxy-3′-Thionucleoside Triphosphates: Syntheses and Polymerase Substrate Activities. Supporting Information.
9. Elena V. Bichenkova, Zhaolei Lang, Xuan Yu 1, Candelaria Rogert 2, Kenneth T. Douglas (2011), DNA-mounted self-assembly: New approaches for genomic analysis and SNP detection. Biochimica et Biophysica Acta, 1809 1-23.
10. Miller, G. P., Silverman, A. P. & Kool, E. (2008). New, stronger nucleophiles for nucleic acid-templated chemistry: Synthesis and application in fluorescence detection of cellular RNA. Bioorganic & medicinal chemistry, 16(1), 56-64.
11. Franzini, R. M. and Kool, E. (2008). 7-Azidomethoxy-coumarins as profluorophores for template nucleic acid detection. ChemBioChem 9: 2981-2988.
12. Franzini, R. M. and Kool, E. (2009). Efficient nucleic acid detection by template reductive quencher release. J. Am. Chem. Soc. 131: 16021-16023.
13. Silverman, A. P. and Kool, E. (2005). Quenched autoligation probes allow discrimination of live bacterial species by single nucleotide differences in rRNA. Nucleic Acids Res. 33: 4978-4986.
14. Sando, S, and Kool, E. (2002). Nonenzymatic DNA ligation in Escherichia coli cells. Nucleic Acids Res. Supplement No. 2: 121-122.
15. Abe, H. and Kool., E. (2006). Flow cytometric detection of specific RNAs in native human cells with quenched autoligating FRET probes. Proc. Natl. Acad. Sci. USA 103: 263-268.

What is claimed is:

1. A method for exponentially amplifying a specific target nucleic acid sequence, comprising:
(a) contacting the target nucleic acid sequence with a first forward primer nucleic acid, a second forward primer nucleic acid, a first reverse primer nucleic acid and a second reverse primer nucleic acid under conditions wherein the primer nucleic acids specifically anneal with the target nucleic acid sequence;
wherein one forward primer nucleic acid has a thermally stable first bond-forming reactive moiety and the other forward primer nucleic acid has a thermally stable second bond-forming reactive moiety;
wherein one reverse primer nucleic acid has a thermally stable first bond-forming reactive moiety and the other reverse primer nucleic acid has a thermally stable second bond-forming reactive moiety;
wherein the first forward primer nucleic acid and the second forward primer nucleic acid are annealed to the target nucleic acid sequence such that the reactive moiety of the first forward primer nucleic acid and the reactive moiety of the second forward primer nucleic acid are juxtaposed;
wherein the first reverse primer nucleic acid and the second reverse primer nucleic acid are annealed to the target nucleic acid sequence such that the reactive moiety of the first reverse primer nucleic acid and the reactive moiety of the second reverse primer nucleic acid are juxtaposed;
wherein the reactive moiety of the first forward primer nucleic acid forms a chemical bond with the reactive moiety of the second forward primer nucleic acid to form a first ligation product, and the reactive moiety of the first reverse primer nucleic acid forms a chemical bond with the reactive moiety of the second reverse primer nucleic acid to form a second ligation product;
whereby the first ligation product forms a duplex with the target nucleic acid sequence and the second ligation product forms a duplex with the target nucleic acid sequence;
(b) further comprising thermally disrupting the duplexes to form target nucleic acid sequences;
and repeating step (a).

2. The method according to claim 1, wherein the first reactive moiety is at a 3′ terminus of the first forward primer and the second reactive moiety is at a 5′ terminus of the second forward primer.

3. The method according to claim 1, wherein the first reactive moiety is at a 3′ terminus of the first reverse primer and the second reactive moiety is at a 5′ terminus of the second reverse primer.

4. The method according to claim 1, wherein the first reactive moiety is selected from an electrophile and a nucleophile and the second reactive moiety is selected from an electrophile and a nucleophile, wherein when one reactive moiety is an electrophile, the other reactive moiety is a nucleophile.

5. The method according to claim 1, wherein the first and second reactive moieties are selected from the group consisting of phosphorodithioate, phosphorotrithioate, 2′,3′-cyclic phosphate, amino-deoxyribonucleosides, thiol, amino, hydrazine, hydrazide, bromide, iodide, chloride, maleimide, dabsylate, pyridyldisulfide, tosylate, alkyne, isothiocyanate, cyclooctyne, NHS ester, imidoester, PFP ester, alkyl azide, aryl azide, isocyanate, nitrophenyl mono- or di-ester and epoxy wherein when one of the reactive moieties is selected from the group consisting of phosphorodithioate, phosphorotrithioate, 2′,3′-cyclic phosphate, amino-deoxyribonucleosides, thiol, amino, hydrazine and hydrazide, the other reactive moiety is selected from the group consisting of bromide, iodide, chloride, maleimide, dabsylate, pyridyldisulfide, tosylate, alkyne, isothiocyanate, cyclooctyne, NHS ester, imidoester, PFP ester, alkyl azide, aryl azide, isocyanate, nitrophenyl mono- or di-ester and epoxy.

6. The method according to claim 4, wherein: a) the thermally stable electrophile is selected from an alkyl-halide moiety; a maleimide moiety;
b) the thermally stable nucelophile is thiol;
c) the thermally stable electrophile is bromoacetamide and the thermally stable nucleophile is thiol; or
d) the thermally stable electrophile is a haloacetamide moiety.

7. The method according to claim 6, wherein the haloacetamide moiety is a bromoacetamide moiety.

8. The method according to claim 1, wherein at least one forward or reverse primer is conjugated to a detectable group.

9. The method according to claim 8, wherein at least one detectable group is selected from the detectable groups listed in Table 1.

10. The method according to claim 1, wherein one forward or reverse primer nucleic acid comprises a FRET donor fluorophore and the other forward or reverse primer nucleic acid comprises a FRET acceptor fluorophore, and wherein the method further comprises detecting the ligation product by FRET.

11. The method according to claim 2, wherein the FRET donor fluorophore is FAM and the FRET acceptor fluorophore is Texas Red.

12. A reagent for exponentially amplifying a target nucleic acid sequence comprising:
a) a first forward primer nucleic acid having a thermally stable first bond-forming reactive moiety;
b) a second forward primer nucleic acid having a thermally stable second bond-forming reactive moiety;
c) a first reverse primer nucleic acid having a thermally stable first bond-forming reactive moiety; or
d) a second reverse primer nucleic acid having a thermally stable second bond-forming reactive moiety.

13. The reagent according to claim 12, wherein the bond-forming reactive moiety is an electrophile or a nucleophile.

14. The reagent according to claim 13, wherein: a) the electrophile is selected from the alkyl-halide moiety and a maleimide moiety;
b) the electrophile is a haloacetamide moiety;
c) the electrophile is a bromoacetamide moiety; or
d) the nucleophile is thiol.

15. The reagent according to claim 12, wherein the bond-forming reactive moiety is selected from the group consisting of phosphorodithioate, phosphorotrithioate, 2′,3′-cyclic phosphate, amino-deoxyribonucleosides, thiol, amino, hydrazine, hydrazide, bromide, iodide, chloride, maleimide, dabsylate, pyridyldisulfide, tosylate, alkyne, isothiocyanate, cyclooctyne, NHS ester, imidoester, PFP ester, alkyl azide, aryl azide, isocyanate, nitrophenyl mono- or di-ester and epoxy.

16. The reagent according to claim 15, wherein: a) the bond-forming reactive moiety is at a 3′ terminus; or
b) the bond-forming reactive moiety is at a 5′ terminus.

17. The reagent according to claim 12, further comprising a detectable group.

18. The reagent according to claim 17, wherein the detectable group is selected from the group consisting of the detectable groups listed in Table 1.

19. The reagent according to claim 17, further comprising a FRET donor fluorophore or a FRET acceptor fluorophore.

20. A kit for exponentially amplifying a target nucleic acid sequence, comprising a reagent according to claim 12, wherein the first bond-forming reactive moieties are capable of forming a chemical bond with the second bond-forming reactive moieties.
